(19) United States
(12) Patent Application Publication
(10) Pub. No.: US 2020/0254007 A1
(43) Pub. Date: Aug. 13, 2020

(54) METHOD OF PRODUCTION OF A BRANCHED ORGANOSILICONE POLYMEROUS POLYNUCLEAR ADSORBENT OF HIGH MOLECULAR TOXINS AND THIS ADSORBENT

(71) Applicant: HEALTH PRODUCT GROUP SP. Z O.O., Warsaw (PL)

(72) Inventors: Vadym KOZLOVSKY, Kyiv (UA); Juriy TOLCHEYEV, Kyiv (UA)

(21) Appl. No.: 16/753,790

(22) PCT Filed: Feb. 19, 2018
(86) PCT No.: PCT/IB18/51005

§ 371 Date: Apr. 5, 2020

(30) Foreign Application Priority Data
Oct. 13, 2017 (UA) 201709921

(51) Int. Cl. A61K 31/80; A61P 39/00

(52) U.S. Cl. CPC A61K 31/80; A61P 39/00

(57) ABSTRACT

The synthesis of an organosilicone adsorbent, which can be used in medicine as an enterosorbent for the removal of organic and toxic metabolites from the body obtains a new branched organosilicone polymerous polynuclear adsorbent of high molecular toxins. According to the analysis by Si²⁹NMR method, the adsorbent contains superpositions of at least three silicon signals with values of chemical shifts −80 ppm, −100 ppm, −115 ppm, and ratio of their integral intensities close to 2:2:1. This adsorbent has a high adsorption activity, and the synthesis of its obtaining can reduce labour costs, increase output of the target product in manufacture and reduce their self-cost/net-cost.

### CROSS-REFERENCE TO RELATED APPLICATIONS

See also Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The invention relates to the synthesis of an organo silicone adsorbent, which can be used in various sectors of the national economy, especially in medicine as an enterosorbent for the removal of organic and toxic metabolites from the body, including in various types of pathology of the gastrointestinal tract for the internal, as well as for the external use.

### 2. Description of Related Art Including Information Disclosed Under 37 CFR 1.97 and 37 CFR 1.98.

The interest in the use of sorbents for medical purposes has increased dramatically since the 80s of the last century. This was preceded by studies of Greek scientists who demonstrated the possibility of effective removing toxic products from the body, formed in it as a result of diseases, by cleaning the blood with the help of activated charcoal. The obtained results gave a powerful push to the development of new methods of treatment and the development of new types of sorbents.

In general, sorbents are classified according to the form, chemical nature of the material, texture, type of interaction with the sorbate. The texture is associated with the composing of the primary particles (globules) of the material and thus the formation of pores of a certain size. Each sorbent is characterized by the pore size, pore volume, specific surface, where adsorption occurs—concentration of the substance of the adsorbate (toxin) in the porous space and on the outer geometric surface of the sorbent.

The sorption capacity of porous materials is known to be largely determined by the size of the surface, available for adsorbate molecules, and depends on the exclusive effect. With the growth of the specific surface and, correspondingly, with the decrease in the average pore size of the sorbent, the exclusive effect intensifies, which leads to the appearance of a maximum on the curve of adsorption dependence (per unit of weight of the sorbent) on the pore size.

The classical method for determining these parameters is based on the measurement of the specific surface (S), for example, on nitrogen adsorption, the specific pore volume V—by benzol capillary condensation, the average pore diameter (D) is calculated by formula D=4V/S.

Activated charcoal is of widespread as a sorbent. (Vidal Formulary. Medicinal Products of Russia.—M.: OVPEE—AstraPharm Service, 2000—P. 3-8). It is convenient to use, since it is manufactured in the form of tablets, powder and is used in the chemical, food, pharmaceutical and medical industries.

However activated charcoal has significant disadvantages: moderate adsorption activity and low selectivity of sorption action, such as in relation to cholesterol, bilirubin, urea, and uric acid, which are usually removed from the body by organs of the endocrine system.

When applying it, constipation, vomiting, inhalation of coal dust in the lungs are possible; during chronic use—hypovitaminosis, impaired suction from the gastrointestinal tract of nutrients are possible.

Activated charcoal is contraindicated in case of ulcerous lesions of the gastrointestinal tract, stomach bleeding and for children.

There is also another enterosorbent, the product of non-linear condensation of methylsilicic acid (in the chemical meaning—polymethylsiloxane polyhydrate), known under the trade name Enterosgel (TM).

International Patent Application WO/2011/075095 discloses the method of preparation of hydrogel methylsilicic acid from methyltriethoxysilane (MTES) and aqueous ethanol solution with a volume fraction of ethyl alcohol from 60 to 96.5%, which is treated with a mixture of hydrochloric acid and purified water.

The hydrolysis of MTES is carried out in the presence of an acid catalyst, followed by alkaline treatment at temperature of 16-30° C., with the stand of the reaction mass (for maturation for at least 7.5 h), by grinding the obtained alcogel methylsilicic acid, followed by washing it with treated water at a rate of 2-4 1 per hour before receiving hydrogel.

However, this method of synthesis also has some disadvantages. Sufficient volumes of alcohol are required for the hydrolysis, a long process of “maturation” of gel, high water flow during washing, and the possibility of finding alcohol in pores of the finished product limit the medical use of the sorbent.

From Patent RU 2111979, a method for the synthesis of polymeric compounds of methylsilicic acid is known with the following chemical formula

{(CH₃SiO_{1.5})nH₂O}., where n=44-49,

as an adsorbent of median-molecular metabolites.

Medium-molecular metabolites are substances with a molecular weight of about 500-5000 Da. As a rule, these products are products of disintegration/decay of proteins, which plays an important role in the phenomena of intoxication.

However, macromolecular compounds—bacterial toxins, viruses make not less, but more often and more important component of intoxication.

As it is known from the aforementioned patent, hydrogel of methylsilicic acid (HGMSA) is synthesized by polycondensation of solution of sodium methylesiliconate (or potassium) in concentration from 1.75 to 2.30 mol/1 by adding solution of strong acid (for example, HCl or H2SO4) at temperature of the reaction mixture from +10 to +35° C. until the formation of hydrogel, which, after standing for 30-90 minutes (the “maturation” process), is pulverized and then activated by the action of a dilute solution of strong acid at concentration from 0.04 to 0.15 g·equ/1 followed by washing with water to neutralize the reaction.

The disadvantage of this well-known method of obtaining HGMSA is that it does not take into account different amount of methylsiliconate sodium (potassium) and strong acid, necessary for the reaction, which makes this method poorly! ! reproducible, and the product is hardly suitable for adsorption of high molecular toxicants, which is of great importance in the medical use.

Patent RU 2293744 describes the process of synthesizing a compound which is referred to as 1,1,3,3-tetrahydroxy-1,3-dimethyldisiloxane polyhydrate, and which corresponds to the formula:

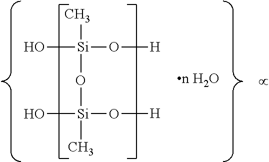

where n from 88 to 98

The essence of the method of obtaining this polyhydrate is the interaction of the original chemical raw materials—“ . . . alkaline solution of dioxymethylsilicic acid sodium formula CH₃Si(OH)₂ONa with density 1.16-1.19 g/cm3 (20 fractions) with sulfuric acid (specific weight 1.195-1.205 g/cm3) (6 fractions), cooled to temperature of 0-5° C., stirring for 90 minutes”.

Meanwhile, neither from the description of this patent nor from another well-known source of the state of art know this reagent! or show the way of obtaining it/the one.

All known publications, related or connected with the subject/topic of silicone hydrogels, are directly or indirectly referred to works of the founder of the school of silicon organic polymers, Academician K. Andrianov and other authors.

In all known sources of information, it is stated that predictable silicones of mono-organosilicones have a complex structure, dependent on the method of preparation, and are polymeric compounds, but not at all monomers with the formula CH₃Si(OH)₂ONA.

Upon completion of the reaction and maturation of the target product, the resulting mixture is washed with water in order to remove unreacted dioxymethyl silicon sodium from pores, which is neutralized by washing at room temperature with solution of sulfuric acid with a specific weight of 1.001 to pH of rinsing water, equal to 4.0, and then with purified water to pH of washing water 5.0-7.0, and pulverized.

The essence of the invention under Patent RU 2293744 is confirmed by Examples 1-3 and the data of the elemental composition, given in the description.

However, according to publication of K. Andrianov, Element Organic Chemistry Methods. Silicon. M.: Nauka, 1968, pp. 550-551, 602-603, there is the method of preparation of methyl siliconate sodium by hydrolyzing methyl trichlorosilane by excess of aqueous alkaline solution. It is assumed that in this case polymers of the following kind are formed:

“Similar phenomena occur during the hydrolysis of methyl trichlorosilane by aqueous alkaline solution, taken with excess (for example, 4 mol per 1 mol of methyl trichlorosilane). In this reaction, block is sodium ions, which form in polymer silanolate groups, which interfere with the reaction between chains:

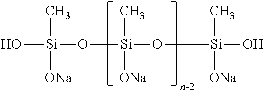

The authors of the claimed invention consider that the compound, protected by Patent RU 2293744 cannot be obtained in principle.

As shown by the authors' own experiments, it is impossible to obtain, in such a well-known way, the target product—gel, since stirring within 90 minutes does not allow it to be formed—to “precipitate out”, that is to turn into the gel state, but on the contrary, there is a paste/pasty amorphous mass, the output of which after pulverising and decanting cannot be 96%.

In connection with the above, there is a task of obtaining new adsorbents on the basis of organosilicones, which would allow to increase their adsorption activity, reduce labour costs, increase the output of the target product in manufacturing/while producing, reduce their self-cost/net-cost.

### BRIEF SUMMARY OF THE INVENTION

Assigned task is solved by creating a method for obtaining of a new branched organosilicone polymer polynuclear silicon compound (polymethylsilsesquioxane), which according to the analysis by Si²⁹NMR method, contains at least three types of silicon atoms, characterized by the presence of a superposition of three silicon signals with values of chemical shifts −80 ppm, −100 ppm, −115 ppm, and the ratio of their integral intensities close (but not necessarily) to 2:2:1 of the general empirical formula, ({CH₃SiO(OH)}x·(CH₃SiO_{1.5})y·(2H₂O)z)ₙ,

where x—from 0.1 to 0.9; y—from 0.9 to 0.1; z—from 1.55 to 2.55, n—more than 2

The proposed method provides the process of spatial non-linear growth of the polymeric chain and contributes to formation of a globular structure that ensures formation of pores, and, accordingly, provides adsorption activity to the sorbent in the declared method.

### BRIEF DESCRIPTION OF THE SEVERAL VIEWS OF THE DRAWINGS

FIG. 1 is a graph illustration of infrared spectra confirming the branched organosilicone polymerous polynuclear compound of the invention.

FIG. 2 shows a graph illustration of analysis by the Si²⁹NMR method.

### DETAILED DESCRIPTION OF THE INVENTION

According to the claimed invention, in the beginning of the obtaining process of the product of the hydrolysis of methyl trichlorosilane of the general formula (CH₃SiO₂Na)ₙ, is subjected to alkaline treatment until the dissolution process is complete, water is added to achieve a concentration of the product of the hydrolysis of methyl trichlorosilane not less than 250 g/l, which according to the analysis by Si²⁹NMR method, comprises at least three superposition of silicon with values of chemical shifts −18 ppm, −26ppm, −33 ppm. After, processing of the product is carried out by solution of strong acid, ensuring formation of hydrogel, which, after completion of the polycondensation is pulverized and treated with a dilute solution of strong acid, followed by washing with water until the neutral reaction and obtaining the target product, which has the general empirical formula:

({CH₃SiO(OH)}x·(CH₃SiO_{1.5})y·(2H₂)z)ₙ,

where x—from 0.1 to 0.9; y—from 0.9 to 0.1; z—from 1.55 to 2.55, n—more than 2
and which according to the analysis by Si²⁹NMR method contains superpositions of at least three silicon signals with values of chemical shifts - 80 ppm, - 100 ppm, - 115 ppm, and ratio of their integral intensities close to 2:2:1.

It is advisable, after treatment with alkaline before completion of the dissolution process, to add water in ratio 1:0.3-0.4:0.7-2, and treatment with a strong acid solution to carry out in a volumetric ratio of components 5-10 to 1.

In addition, crushed hydrogel can preferably be treated with a dilute solution of strong acid, having temperature not higher than 15° C. and concentration from 0.02 to 0.2 g·equ/1.

As conducted experiments show, as a result of synthesis at given temperature regime of the reaction mixture, the target product with the following physical and chemical properties, presented in Table 1 with a high sorption capacity to high molecular toxins (toxic agents), is formed.

TABLE 1
Influence of synthesis temperature on the appearance
of the target product and its output

| | Value of temperature | | |
|---|---|---|---|
| | of the synthesis | Target | Target |
| No. of | reaction of the | product | product |
| experiment | target product | output, % | properties |
| 1 | 35 | — | product has no form |
| 2 | 30 | — | |
| 3 | 25 | 30 ± 5 | amorphous mass |
| 4 | 20 | 60 ± 5 | amorphous mass |
| 5 | 15 | 70 ± 5 | Amorphous mass |
| 6 | 10 | 70 ± 5 | weakly gel-like mass |
| 7 | 9.5 | 78 ± 5 | Gel-likr mass |
| 8 | 8 | 80 ± 5 | Stable gel-like mass |
| 9 | 7 | 80 ± 5 | stable gel-like mass |
| 10 | 6 | 85 ± 5 | stable gel-like mass |
| 11 | 5.5 | 80 ± 5 | stable gel-like mass |
| 12 | 5 | 80 ± 5 | stable gel-like mass |
| 13 | 0 | 80 ± 5 | Stable gel-like mass |

Compounds, synthesized according to the claimed method, are also a second aspect of the protection of the present invention.

Mentioned synthesized compounds have been analysed by Si²⁹NMR method, for the content of water, silicon, carbon and hydrogen.

The adsorption activity (A) of the obtained target product has been determined by their ability to absorb Congo red and methyl orange from aqueous solution, as well as high molecular bacterial toxin.

Determination of the adsorption activity has been carried out according to methods, described in Patent RU 2293744.

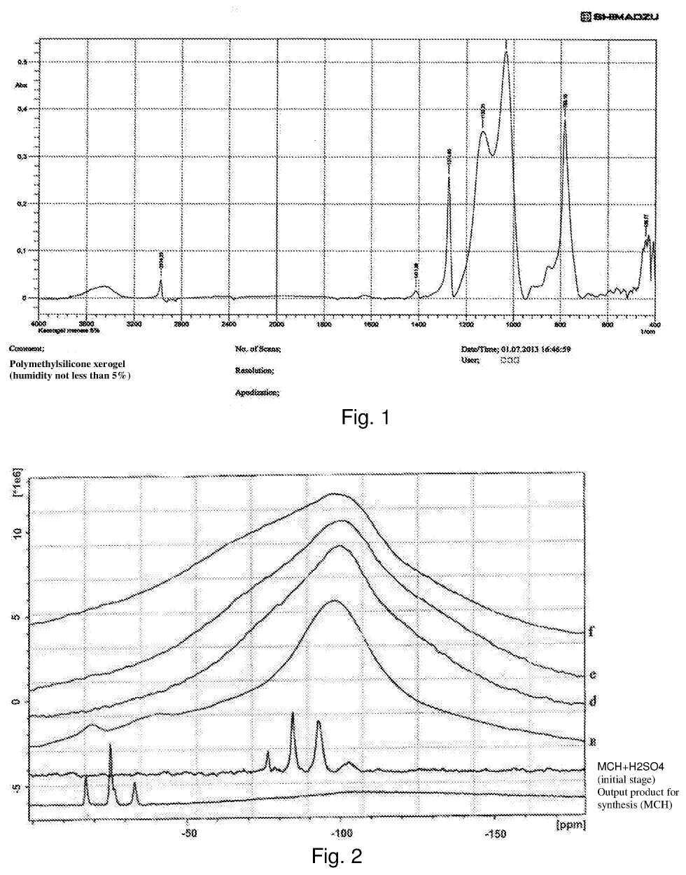

Below, specific Examples of preferred embodiments of the synthesis of the target product with reference to the figure of drawings are given, where FIG. 1 provides experimental IR spectra of dehydrated hydrogels, obtained in accordance with the invention in the frequency range 400-1400⁻¹, where valent fluctuations of groups Si—O(Si) are manifested, and FIG. 2—data of the analysis of the compound, obtained according to the claimed technique by Si²⁹NMR method.

### Example 1

100 liters of water were poured into the reactor and 12 liters of methyltrichlorosilane (MTCS) were added (about 15.24 kg) during stirring for 1 hour. The mixture was stirred for 30 minutes with the aid of a stirrer and then hydrochloric acid was separated by filtration during 15 min. After that, the cycle was repeated. In this process, the reaction mixture was heated to temperature of 60-70° C. The washing of the product, formed as a result of two cycles, was carried out in the same mode until the acidity of washing waters was equal to pH 5.0. 12.4 kg of the product of the hydrolysis of MTCS was obtained (output of about 80% by weight)

The resulting product of the hydrolysis of trichloromethylsilane was subjected to treatment with alkaline—sodium hydroxide—not less than 97% in such a ratio of components' masses: for 1 fractional of the product of the hydrolysis not less than 10 weight fractions of sodium hydroxide until completion of the dissolution process, followed by addition of water to achieve concentration of the product of the hydrolysis of methyltrichlorosilane not less than 250 g/l.

31 kg of hydrated product of the hydrolysis of MTCS, containing according to data of Si²⁹NMR at least 3 types of silicon atoms and characterized by the presence of a superposition of three silicon signals with values of chemical shifts −18 ppm, −26 ppm, −33 ppm, and ratio of their integral intensities close to 2:2:1 is obtained.

After that, the resulting product was added with a solution of sulfuric acid (not less than 30%) of 3.4 liters. After 1.3 minutes, gel product (sedimentation) was obtained. After pulverizing and washing from acid to neutral washings, 37.2 kg of hydrogel polymeric organosilicone polynuclear adsorbent was obtained, which according to the analysis by Si²⁹NMR method, comprises superposition of at least three silicon signals with values of chemical shifts −80 ppm, −100 ppm, −115 ppm, and ratio of their integral intensities close to 2:2:1.

The resulting product had a gelled/gel form of almost white colour; not soluble in water and organic solvents; had pH equal to 6.0; dry residue was 8.88%; silicon content was 3.94%; along with that adsorption activity was 3.0 μmol/g.

### Example 2

200 liters of water were poured into the reactor and 25 liters of methyl trichlorosilane (MTCS) (about 31 kg) were added during stirring for 1 hour. The mixture was stirred for 50 minutes with the aid of a stirrer and then hydrochloric acid was separated by filtration for 30 min. After that, the cycle was repeated. In this process, the reaction mixture was heated to temperature of 60-70° C. The washing of the product, formed as a result of two cycles, was carried out in the same mode until the acidity of rinsing waters was equal to pH 5.5. 22 kg of the product of the hydrolysis of MTCS was obtained (output of about 70% by weight)

The resulting product of the hydrolysis of trichloromethylsilane was subjected to treatment with alkaline—sodium hydroxide—not less than 97% in such a ratio of components' masses: for 1 fractional of the product of the hydrolysis not less than 10 weight fractions of sodium hydroxide until completion of the dissolution process, followed by addition of water to achieve concentration of the product of the hydrolysis of methyl trichlorosilane not less than 250 g/l.

60 kg of hydrated product of the hydrolysis of MTCS, containing according to data of NMR Si²⁹ at least 3 types of silicon atoms and characterized by the presence of a superposition of three silicon signals with values of chemical shifts −18 ppm, −26 ppm, −33 ppm, and ratio of their integral intensities close to 2:2:1 is obtained.

After that, the resulting product was added with a solution of sulfuric acid (not less than 30%) of 5 liters. After 1.5 minutes, gel product (sedimentation) was obtained. After pulverizing and washing from acid to neutral washings, 72.2 kg of hydrogel was obtained from polymerous organosilicone polynuclear adsorbent, which according to the analysis by Si²⁹NMR method, comprises superposition of at least three silicon signals with values of chemical shifts −80 ppm, −100 ppm, −115 ppm, and ratio of their integral intensities close to 2:2:1.

The resulting product had a softgell form of almost white colour; not soluble in water and organic solvents; had pH equal to 6.3; dry residue was 8.9%; silicon content was 3.90%; along with that adsorption activity was 2.8 μmol/g .

Thus, the indicated method allows to obtain an end- product, of a stabile composition, and which according to the analysis by Si²⁹NMR method contains superpositions of at least three silicon signals with values of chemical shifts −80 ppm, −100 ppm, −115 ppm, and ratio of their integral intensities close to 2:2:1.

The chemical structure of the obtained branched organosilicone polymerous polynuclear compound is confirmed by infrared spectra, obtained in the frequency range from 400 to 1400 cm⁻¹, given in FIG. 1, which show valent fluctuations of groups Si—O(Si), by Si²⁹NMR method, which made it possible to distinguish between changes in the nature of the environment of resonant silicon atoms, a qualitative reaction to silicon, the method of elemental analysis, which indicates ratio of elements in the structure.

To show experimentally the structure of the globular matrix of the product, pore solvent water was removed by heating at 130° C. and recorded by IR. IR spectra in range 400-1400⁻¹ were recorded by Shimadzu spectrophotometer.

The resulting product had a slightly gel/gelled form of almost white colour; not soluble in water and organic solvents; had pH equal to 6.3; dry residue was 8.9%; silicon content was 3.90%; herewith adsorption activity was 2.8 μmol/g.

As can be seen from FIG. 1, the experimental spectrum records three intensive absorption bands at 1003 cm⁻¹, 1132 cm⁻¹ and 1274 cm⁻¹. Frequencies in range 2800-3750 cm⁻¹ relate to valent fluctuations of Si—OH groups. The presence in the spectrum of hydroxyl groups indicated the possibility of formation along with chemical bonds, intraglobular bonds. It is known that absorption bands of Si—O bonds are in the range 900-1000 cm⁻¹, in three-dimensional absorption structures Si—O-bonds are observed in range 1050-1200 cm⁻¹.

According to the data obtained from the analysis by Si²⁹NMR method, shown in FIG. 2, the investigated compound contains at least three types of silicon atoms, characterized by the presence of superposition of three silicon signals (d and e—synthesized compound, f—xerogel of synthesized compound, and b—model kuban—polymer, which contains a Si—O—Si dimer as a monomer component and is characterized by a single silicon signal) with values of chemical shifts −80 ppm, −100 ppm, −115 ppm, and ratio of their integral intensities close to 2:2:1.

Thus, it is confirmed that there is a dependency between the presence of three types of silicon atoms in the basic substance for synthesis of the final product, which indicates polynucleosis and branching, and allows the polymerization reaction to be carried out precisely at the expense of these groups and the structure of the final product (more negative values the chemical shift in comparison with the basic product (the product of the hydrolysis of methyl trichlorosilane) indicate polymerisation). Changes in the concentration parameters of the reaction, starting with the hydrolysis of MTCS, and ending with gel, will lead to a change in the structure of the finished product.

Studies of infrared spectra of the obtained compounds have been carried out. Frequencies in range 2800-3750cm⁻¹ of infrared spectra relate to valent fluctuations of Si—OH groups and indicate the possibility of formation of intraglobular bonds along with the chemical bonds, that confirms the branched globular structure of the claimed compound.

More over, the globular structure is confirmed by studies in terms of determination of the pore size and specific surface area, and the adsorption activity of benzol, which are shown in Table 2.

TABLE 2

| | Capacity for benzol, g/cm³ |
|---|---|
| Sample No. 1: synthesized organosilicone | 0.73 |
| Sample No. 2 synthesized organosilicone | 0.83 |
| activated charcoal (control, GOST) | 0.46 |

The chemical structure of the claimed substance is also proved, using the elemental analysis by method of laser analysis of the elemental composition with the use of LEA-S500 apparatus, the results of which are given in Table 3.

As follows from Table 3, the proportion of silicon, carbon and hydrogen in the adsorbent according to the invention coincides with the calculated values of these figures. Thus, using the elemental analysis, the chemical structure of the declared adsorbent has been confirmed.

TABLE 3
Elemental composition of organosilicone sample

| Element | C | Si | H |
|---|---|---|---|
| Found, % | 20.0 | 40.5 | 5.0 |
| Calculated, % | 18.0 | 41.8 | 4.5 |

Thus, ratio of elements: C 0.49: Si 1: H 0.12 corresponds to the declared empirical formula.

To confirm affinity of high molecular toxicagens, measurement of the adsorption activity for high molecular substances—immunoglobulin G (more than 100,000 Da) and bacterial toxin (more than 10,000 Da) was performed.

The results are shown in Table 4.

TABLE 4
Comparative experimental data on the study of properties
of the prototype and the claimed technical solution

| | Name of indicators | Prototype | | Claimed technical | |
|---|---|---|---|---|---|
| No. | compared | indicator | | solution indicator | |
| 1 | Methylene blue | 3.2 | μmol/g | 3.2 | μmol/g |
| 2 | Immunoglobulin G | 182.2 | μmol/g | 336.4 | μmol/g |
| 3 | Bacterial toxin | 6.22 | μmol/g | 9.13 | μmol/g |

As follows from the results of Table 4, the branched polymerous polynuclear adsorbent exhibits more signified activity in relation to high molecular compounds and exceeds such activity for the prototype in 1.5-1.83 times.

1. A method for obtaining of a branched polymerous polynuclear adsorbent of high molecular toxins, according to which the product of the hydrolysis of methyl trichlorosilane of a general formula (CH₃SiO₂Na)ₙ, is subjected to alkaline treatment until completion of the dissolution process, water is added to achieve a concentration of the product of the hydrolysis of methyl trichlorosilane not less than 250 g/l, which according to the analysis by Si²⁹NMR method, comprises at least three superposition of silicon with values of chemical shifts 18 ppm, 26 ppm, 33 ppm, the product is processed with a solution of strong acid, ensuring formation of hydrogel, which, after completion of the polycondensation process is pulverized and treated with a dilute solution of strong acid, followed by washing with water until the neutral reaction and obtaining of the target product, which has the general empirical formula:
({CH₃SiO(OH)}x·(CH₃SiO_{1.5})y·(2H₂O)z)ₙ,
where x—from 0.1 to 0.9; y—from 0.9 to 0.1; z—from 1.55 to 2.55, n—more than 2 and which according to the analysis by Si²⁹NMR method contains superpositions of at least three silicon signals with values of chemical shifts −80 ppm, −100 ppm, −115 ppm, and ratio of their integral intensities close to 2:2:1.

2. The method of claim 1, by which after treatment with alkaline till completion of the dissolution process, water is added in ratio 1:0.3-0.4:0.7-2.

3. The method of claim 1, by which treatment with strong acid solution is carried out in a bulk ratio of components 5-10 to 1.

4. The method of claim 1, by which pulverized hydrogel is treated with a dilute solution of strong acid, having temperature not higher than 15° C. and concentration from 0.04 to 0.15 g·equ/1.

5. A composition of branched polymerous polynuclear adsorbent of high molecular compounds of the general empirical formula,
({CH₃SiO(OH)}x·(CH₃SiO_{1.5})y·(2H₂O)z)ₙ,
where x—from 0.1 to 0.9; y—from 0.9 to 0.1; z—from 1.55 to 2.55, n—more than 2 which is obtained by the method according to paragraphs 1-4 and which according to the analysis by Si²⁹NMR method contains superpositions of at least three silicon signals with values of chemical shifts −80 ppm, −100 ppm, −115 ppm, and the ratio of their integral intensities close to 2:2:1.
